(19) United States
(12) Patent Application Publication
(10) Pub. No.: US 2019/0390245 A1
(43) Pub. Date: Dec. 26, 2019

(54) CYTOTOXICITY TEST METHOD FOR MEDICAL DEVICE

(71) Applicant: ALCON INC., Fibourg (CH)

(72) Inventors: Xuemei Liu, Fort Worth, TX (US); Charlon Tolliver, Forth Worth, TX (US); Denise Rodeheaver, Fort Worth, TX (US); Stephen Paul Shannon, Arlington, TX (US); Fan Zhang, Keller, TX (US); Lisa Maddox Walker, Fort Worth, TX (US); Mercedes Salvador-Silva, Mansfield, TX (US); Kenneth Keven Williams, Kennedale, TX (US)

(21) Appl. No.: 16/451,504

(22) Filed: Jun. 25, 2019

Related U.S. Application Data
(60) Provisional application No. 62/690,046, filed on Jun. 26, 2018.

(51) Int. Cl. C12Q 1/04; C12Q 1/02

(52) U.S. Cl. CPC C12Q 1/045; C12Q 1/025

(57) ABSTRACT

A new two-layer modified agar overlay cytotoxicity method for medical device safety assessment are developed. The new two layer modified agar overlay cytotoxicity method has equivalent sensitivity, and greater efficiency compared to the ISO/USP direct contact and agar overlay assays. Assays were carried out in 6-well microplates. Nutrient agar medium with a 0.5% agar concentration was used to provide a base nutrient layer to support cell growth, L929 cells mixed nutrient agar (0.33% agar) were seeded on top of the base agar and cytotoxicity was evaluated after 24 hours per USP. Results demonstrated the two layer modified agar overlay cytotoxicity assay performs as well as the ISO/USP direct contact method with distinct advantages. Results showed that this two layer modified agar overlay method is more promising as compared to the traditional agar overlay and direct contact methods due to the diluted soft agar, avoided potential mechanical damage from test materials, and represents a valuable tool to evaluate medical device cytotoxicity.

This application claims the benefit under 35 USC § 119 (e) of U.S. provisional application No. 62/690,046 filed 26 Jun. 2018, herein incorporated by reference in its entirety.

The invention relates to a method of using a two layer modified agar overlay procedure to assess cytotoxicity for a medical device, such as a contact lens, particularly a silicon hydrogel contact lens.

### BACKGROUND OF THE INVENTION

Medical device nonclinical biocompatibility testing is used to evaluate the risk of adverse effects on tissues from exposure to medical device materials or chemical extracts and/or leachates. For new or modified medical devices, a battery of in vitro and in vivo nonclinical tests is recommended in accordance with relevant international regulatory standards to determine if the device and its material are biocompatible. In vitro cytotoxicity, a key element of these international standards, is a required endpoint of evaluation and assessment for all types of medical devices. The cytotoxicity test is among the many biological evaluations of test substances that utilize in vitro tissue cells to assess adverse effects of medical devices on cell growth, replication, and morphology in the development of medical devices. It allows rapid evaluation, employs standard protocols, produces comparable data, and, due to its sensitivity, enables toxic substances to be identified prior to animal testing. The high sensitivity of in vitro cytotoxicity tests compared to animal studies might due to the direct exposure of cells to the material being tested and the absence of the protective mechanisms that assist cells in vivo.

Generally, two types of qualitative direct contact cytotoxicity tests for medical device regulatory submissions are recommended: a) agar overlay/diffusion and b) direct contact method. These two methods assess the test material through indirect contact by diffusion of material extracts/eachables through agar overlaying the cells or direct contact of the test material with cells. The ISO 10993-5 (2009) qualitative agar diffusion/overlay assay is appropriate for high density materials. This assay is conducted by adding a thin layer of nutrient-supplemented agar over the sub-confluent L929 cells and the test material (a solid sample or an extract of the test material on filter paper) is placed on top of the agar layer covering around 10% of the surface of the cell culture. L929 cells are exposed to a concentration gradient of the leachates from the test material due to the diffusion with highest concentration and by the strongest effects under the test materials. The cushioning effect of an agar layer protects the L929 cells from the potential mechanical damage by the test materials movement. Cells are examined at 24 hours for signs of cytotoxicity and a zone of malformed, degenerative, or lysed cells under and around the test material indicates cytotoxicity. The disadvantage of agar overlay/diffusion assay is that potential cytotoxic leachates may not be able to diffuse across the agar; thus, the cells may not be fully exposed to the test substance.

The direct contact procedure is recommended for low density materials, such as contact lens polymers, per ISO 10993-5 to better reflect clinical use. In this method, a piece of test material is placed directly onto sub-confluent cultures of L929 fibroblast cells. During incubation, the leachable chemicals in the test material can diffuse into the culture medium and contact the cell layer. At the end of the incubation, cell culture medium was removed and replaced with the stain. Reactivity of the test sample is indicated by malformation, degeneration, and lysis of cells under and beyond the test material. The major disadvantage of this method is the movement of the test materials during the study is not fully controlled. Originally, it was suggested that the test material movement was associated with the volume of culture medium used during the incubation. However, neither decreasing the volume of media nor placing a weight on top of the test material (such as contact lenses) can completely prevented test article movement. In addition, using the direct contact method, the test material is either placed directly on the cells without the cushioning effect of an agar layer, or the cells are cultured on the surface of the medical device itself. Since the cells are not protected by an overlying agar layer, cells in the direct contact are more susceptible to potential mechanical damage by the overlying test substance. The fact that removal of the agar layer may increase assay sensitivity, but this step also leaves the cell surface susceptible to mechanical damage that can be mistaken for chemical damage. In addition, using the direct contact method, the test material is either placed directly on top of the cells which might cause damages due to lacking of nutrient and oxygen/carbon dioxide or the cells are cultured on the surface of the medical device itself, although usefulness of the latter is limited by the ability of the cells to adhere to the test material.

Due to the increased new types of medical devices to be evaluated, there continues to be an important need for better screens to develop a sensitive qualitative method to assess cytotoxicity for a medical device.

### SUMMARY OF THE INVENTION

This invention is directed to a method of measuring the cytotoxicity of a medical device, the method comprising:

a) providing a first agar-nutrition medium to a well plate to form a coating of the first agar-nutrition medium onto a surface of the well plate;

b) placing a mixture of second agar-nutrition medium and a cell onto the top of the first agar-nutrition medium;

c) placing the medical device onto the top of the second agar-nutrition medium mixed with a cell, wherein the first agar-nutrition medium and the second agar-nutrition medium comprising a color dye;

d) incubating the well plate containing the medical device with the first agar-nutrition medium and the mixture of second agar-nutrition medium and a cell;

e) determining size of zone of decolorization around and under the medical device;

wherein the first agar-nutrition medium has a higher agar concentration than the second agar-nutrition medium;

wherein each of the first agar-nutrition medium and the second agar-nutrition medium has the agar concentration less than 2 percent.

This invention is also directed to A method of testing cytotoxicity of a medical device comprising:

a) providing a first agar-nutrition medium to a well plate to form a coating of first agar-nutrition onto a surface of the well plate;

b) placing the medical device onto the top of the first agar-nutrition medium;

c) placing a mixture of second agar-nutrition medium and a cell onto the top of the medical device and the second agar-nutrition medium to fully cover the medical device, wherein the first agar-nutrition medium and the second agar-nutrition medium comprising a color dye;

d) incubating the well plate containing the medical device with the first agar-nutrition medium and the mixture of second agar-nutrition medium and a cell; and

e) determining size of zone of decolorization around the medical device;

wherein the first agar-nutrition medium has a higher agar concentration than the second agar-nutrition medium;

wherein both the first agar-nutrition medium and the second agar-nutrition medium have the agar concentration less than 2 percentage.

### BRIEF DESCRIPTION OF THE FIGURES

FIG. 1: Schematic representation of the traditional agar overlay method.

FIG. 2: Schematic representation of the two layer modified agar overlay method

FIG. 3: Schematic representation of the two layer embedded modified agar overlay method

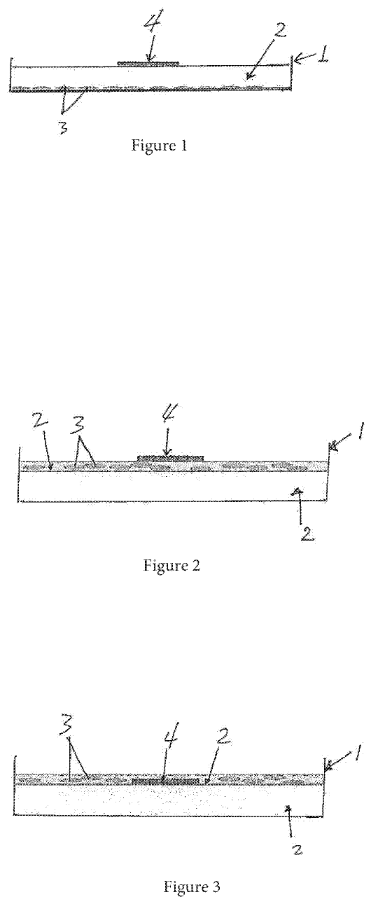

In the FIGS. 1-3, 1 is the well plate; 2 is agar nutrition medium; 3 is cell and 4 is test material

### DETAILED DESCRIPTION

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value.

Similarly, when values are expressed as approximations, by use of the antecedent “about,” it will be understood that the particular value forms another embodiment.

“Cells” are the building blocks of life because they make up all the tissues and parts of our bodies. Cells are herein used in its broadest sense in the art and refers to a living body which is a structural unit of tissue of a multicellular organism, is surrounded by a membrane structure which isolates it from the outside, has the capability of self-replicating, and has genetic information and a mechanism for expressing it. Cells used herein may be naturally-occurring cells or artificially modified cells (e.g., fusion cells, genetically modified cells, etc.). Mouse fibroblast L929 and Chinese hamster lung fibroblast V79-4 cell line are primarily used in this invention.

“Medical device” refers to a device that is introduced temporarily or permanently into a mammal for the prophylaxis or therapy of a medical condition. For example, medical device may be a contact lens or these devices include any that are introduced subcutaneously, percutaneously or surgically to rest within an organ, tissue or lumen. Medical devices may include stents, covered stents such as those covered with polytetrafluoroethylene (PTFE), or expanded polytetrafluoroethylene (ePTFE), synthetic grafts, artificial heart valves, artificial hearts and fixtures to connect the prosthetic organ to the vascular circulation, venous valves, abdominal aortic aneurysm (AAA) grafts, inferior venal caval filters, permanent drug infusion catheters, embolic coils, embolic materials used in vascular embolization (e.g., PVA foams), and vascular sutures. Medical devices may include scaffolds. Scaffolds are defined as three-dimension porous solid biomaterials designed to perform some or all of the following functions: (i) promote cell-biomaterial interactions, cell adhesion, and ECM deposition, (ii) permit sufficient transport of gases, nutrients, and regulatory factors to allow cell survival, proliferation, and differentiation, (iii) biodegrade at a controllable rate that approximates the rate of tissue regeneration under the culture conditions of interest, and (iv) provoke a minimal degree of inflammation or toxicity in vivo. The developing scaffolds with the optimal characteristics, such as their strength, rate of degradation, porosity, and microstructure, as well as their shapes and sizes, are more readily and reproducibly controlled in polymeric scaffolds. Polymer scaffolds can provide mechanical strength, interconnected porosity and surface area, varying surface chemistry, and unique geometries to direct tissue regeneration.

“Contact Lens” refers to a structure that can be placed on or within a wearer's eye. A contact lens can correct, improve, or alter a user's eyesight, but that need not be the case. A contact lens can be of any appropriate material known in the art or later developed, and can be a soft lens, a hard lens, or a hybrid lens. A “silicone hydrogel contact lens” refers to a contact lens comprising a silicone hydrogel material.

A “hydrogel” refers to a polymeric material which can absorb at least 10 percent by weight of water when it is fully hydrated. A hydrogel material can be obtained by polymerization or copolymerization of at least one hydrophilic monomer in the presence of or in the absence of additional monomers and/or macromers or by crosslinking of a prepolymer.

A “silicone hydrogel” refers to a hydrogel obtained by copolymerization of a polymerizable composition comprising at least one silicone-containing vinylic monomer or at least one silicone-containing macromer or a silicone-containing prepolymer.

“Minimal Essential Medium”, also called MEM, is commercially available from Thermo Fisher scientific. The components are: Amino Acids: L-Arginine hydrochloride (126.0 mg/L), L-Cystine 2HCl (31.0 mg/L), L-Histidine hydrochloride-H2O (42.0 mg/L), L-Isoleucine (52.0 mg/L), L-Leucine (52.0 mg/L), L-Lysine hydrochloride (73.0 mg/L), L-Methionine (15.0 mg/L), L-Phenylalanine (32.0 mg/L), L-Threonine (48.0 mg/L), L-Tryptophan (10.0 mg/L), L-Tyrosine disodium salt dihydrate (52.0 mg/L), L-Valine (46.0 mg/L) and Vitamins: Choline chloride (1.0 mg/L), D-Calcium pantothenate (1.0 mg/L), Folic Acid (1.0 mg/L), Niacinamide (1.0 mg/L), Pyridoxal hydrochloride (1.0 mg/L), Riboflavin (0.1 mg/L), Thiamine hydrochloride (1.0 mg/L), i-Inositol (2.0 mg/L), and Inorganic Salts: Calcium Chloride (CaCl2) (anhyd.) (200.0 mg/L), Magnesium Sulfate (MgSO4) (anhyd.) (97.67 mg/L), Potassium Chloride (KCl) (400.0 mg/L), Sodium Bicarbonate (NaHCO₃) (2200.0 mg/L), Sodium Chloride (NaCl) (6800.0 mg/L), Sodium Phosphate monobasic (NaH2PO4-H2O) (140.0 mg/L), and Other component: D-Glucose (Dextrose) (1000.0 mg/L),

“1×MEM” means regular MEM used for cell culture.

“2×MEM” means concentrated (2 times) regular MEM. Usually, agar is prepared in water so in order to have 1×MEM agar, concentrated medium has to use to reach final 1x MEM.

“Agar-nutrition medium” refers to a mixture of agar and nutrition medium. Agar may have a certain predetermined concentration and nutrition medium is the minimal essential medium (MEM) was supplemented with 2 mM L-glutamine, 100 U/mL penicillin and 100 mg/mL streptomycin, and either 10% (v/v) fetal bovine serum (FBS) or 5% (v/v) FBS (designated as 1×MEM10FBS and 1×MEM05FBS respectively). The fetal bovine serum (FBS) concentration can be either 10% or 5%.

“Zone of decolorization” refers to a zone under, around the test medical device has been decolored because the live cells should be able to pick up color dyes (for example, red dyes), which is added into the culture medium but the dead cells cannot. For example, cells are examined after 24 hours incubation for signs of toxicity and stained with neutral red dye to differentiate between the viable, stressed or lysed cells. Cytotoxic leachates diffuse into the cell layer through the agar, and toxicity is indicated by a loss of viable cells around the test device as evidenced by area devoid of stain under and around the test material. Thus the cytotoxic material should has a decolorized zone formed after incubation. The bigger the decolorized zone, the more potent cytotoxic effect the test material has. A ruler for >1.0 cm is used in this application.

Cytotoxicity testing, a primary requirement of all major standards for biological evaluation of medical devices, uses in vitro cell culture systems to assess endpoints of cellular health such as growth, replication, and morphology following exposure to a test material or extract/leachate of a material. Theoretically, it provides a rapid, standardized, sensitive, and economical means to determine whether a material contains potentially biologically harmful activity or substances. One of the benefits of cytotoxicity testing is that it can be used to evaluate the device raw material components as well as the final device itself, and can be utilized as a screening assessment prior to in vivo laboratory animal or human evaluation. Each cytotoxicity method employs standard protocols, generates comparable data across test materials, and enables rapid evaluation so that the potentially toxic materials or substances can be identified prior to in vivo testing. The high sensitivity of in vitro cytotoxicity tests compared to animal studies might due to the direct exposure of cells to the material being tested and the absence of the biological mechanisms that serve to protect organs and tissues in vivo.

Generally, three types of qualitative cytotoxicity tests for medical device regulatory submissions are recommended: a) elution assay (the extract method), b) agar overlay/diffusion (the indirect contact method), and c) the direct contact method. These methods assess the test material by extracts of the material, indirect contact by diffusion of material extracts/leachables through agar overlaying the cells, or cells in direct contact with the test material (IS010993-5; MHLW; USP). Patient use of the device should be considered when selecting test article preparation as well exaggerated conditions to fully evaluate the leachables and extractables of the device under these conditions. For example, if medical devices have long-term contact with tissues, a direct contact or indirect contact cytotoxicity assay might be required in addition to an extract test.

The direct contact method is also recommended for leachable substances that may be deactivated during extraction or for devices or materials that come in direct contact with tissue. Even though test article preparation, exposure time, and staining steps are different for each qualitative method, all the qualitative methods, in general, use the following criteria: 1) negative controls should not have detectable zone beyond the area under specimen (grade ≤2), and 2) positive controls must have produced a zone extending specimen size ≥1.0 cm (reactivity moderate to severe). The test article meets the ‘pass’ criteria of the test if the biological response is less than or equal to grade 2 (mild).

Agar Overlay/Diffusion Assay.

The ISO 10993-5 (2009) qualitative agar diffusion/overlay assay is appropriate for high density materials and liquids. This assay is conducted by adding a thin layer of nutrient-supplemented agar over the near confluent monolayer of L929 cells, and the test material (a solid sample, or an extract of the test material or liquid placed on filter paper) is placed on top of the agar layer. The cushioning effect of an agar layer protects the L929 cells from the potential mechanical damage by the test material's movement. Cells are examined after 24-hours incubation for signs of toxicity and stained with neutral red dye to differentiate between the viable, stressed or lysed cells. Cytotoxic leachates diffuse into the cell layer through the agar, and toxicity is indicated by a loss of viable cells around the test device as evidenced by area devoid of stain under and around the test material. The disadvantage of agar overlay assay is that potential cytotoxic leachates may bind to the agar and not be able to diffuse across the plate as compared to the liquid medium; thus, the cells are not fully exposed to the test substance. As shown previously, in vitro results for dental filling materials obtained from the agarose overlay test and the erythrocyte lysis test did not correlate well with the in vivo results.

Direct Contact Assay.

The direct contact procedure is recommended for low density materials, such as contact lens polymers, per ISO 10993-5 to better reflect clinical use. In this method, the test material (whole or a representative piece) is placed directly onto sub-confluent cultures of L929 fibroblasts. During incubation, the leachable chemicals in the test material diffuse into the culture medium and contact the cell layer. At the end of the incubation, for example, cell culture medium is removed and replaced with fresh culture medium and supplemented with trypan blue stain.

Reactivity of the test sample is indicated by a clear zone of malformation, degeneration and lysis of cells around the test material and the potential cytotoxicity of each test article is graded according to the agar overlay assay. The disadvantage of this method is movement of the test materials during the study cannot be controlled. Since the cells are not protected by an overlying agarose layer, cells in the direct contact are more susceptible to potential mechanical damage by the overlying test substance. Thus, removal of the agar layer may increase assay sensitivity, but also leaves the cell surface susceptible to mechanical damage that can be mistaken for chemical damage. Originally, it was suggested that the test material movement was associated with the volume of culture medium used during the incubation. However, it has been shown that neither decreasing the volume of media nor placing a weight on top of the test material (such as contact lenses) can completely prevent test article movement. In addition, using the direct contact method, the test material is either placed directly on top of the cells which might cause damages due to lacking of nutrient and O₂/CO₂ exchange or the cells are cultured on the surface of the medical device itself, although usefulness of the latter is limited by the ability of the cells to adhere to the test material.

This invention is generally directed to develop a new, two layer modified semisolid agar overlay method using mouse fibroblast L929 or Chinese hamster lung fibroblast V79-4 cell line. This method avoids the physical trauma caused by unintentional movement/pressure of the test material to the adhesion cells in the direct contact method. In this two-layer agar method, 2 mL of the 0.5% agar was used as the base agar and 600 μL of L929 cells mixed with 0.33% of agar was seeded on top of the base agar. For example, provide a first agar-nutrition medium containing 0.5% of agar (base agar) to a well plate to form a coating of the first agar-nutrition medium onto a surface of the well plate, then placing a mixture of second agar-nutrition medium containing 0.33% of agar and a cell onto the top of the first agar-nutrition medium; then placing the medical device onto the top of the second agar-nutrition medium. According to the present invention, each of the first agar-nutrition medium and the second agar-nutrition medium has the agar concentration less than 2 percent. Preferably, each of the first agar-nutrition medium and the second agar nutrition medium has the agar concentration less than 1.5 percent, more preferably, less than 1.0 percent, still more preferably, less than 0.75 percent. Even more preferably, first agar-nutrition medium has 0.5% of agar and the second agar-nutrition medium has 0.33% of agar.

This approach could increase overall solubility of the potential leachates on the material as compared to the traditional agar overlay method with 2 mL of 1% agar laid on top of cells. This modified agar overlay method combines the advantages of the traditional agar overlay and direct contact methods, and additionally rules out both key limitations. In addition, it remains a simple, low-cost and maybe used for a broad range of application like medical devices prepared in situ. Therefore, the use of modified agar overlay appears promising for cytotoxicity studies.

### Material and Methods

The international standard cytotoxicity test reference materials A (polyurethane film containing 0.1% zinc diethyldithiocarbamate), B (polyurethane film containing 0.25% zinc dibutyldithiocarbamate), and C (high-density polyethylene sheet), were purchased from Hatano Research Institute (Hatano Research Institute, Food and Drug Safety Center, Hatano, Kanagawa, Japan), punctured to a diameter of 14 mm and ethylene oxide sterilized. Wako plastic discs were acquired from Wako Chemicals (diameter (c): 14 mm, Richmond, Va.). Latex gloves were obtained from Kimberley-Clark (Roswell, Ga.) and cut into 1 cm×1 cm pieces. All testing materials were freshly prepared prior to each experiment. For liquid test articles (e.g. ethanol or Benzalkonium Chloride (BAK)), 100 μL were tested on a diameter of 14 mm paper filter to improve zone formation in agar overlay assays. All the assays were carried out in 6-well microplates (well plates) unless otherwise specified.

L929 Cell Culture Medium and Agar Preparation.

All the cell culture reagents were purchased from Thermo Fisher Scientific (Grand Island, N.Y.) and cell culture containers were purchased from Corning (Corning, N.Y.) unless stated otherwise. The minimal essential medium (MEM) was supplemented with 2 mM L-glutamine, 100 U/mL penicillin and 100 μg/mL streptomycin, and either 10% (v/v) fetal bovine serum (FBS) or 5% (v/v) FBS (designated as 1×MEM_{10FBS} and 1×MEM_{05FBS} respectively). Assay medium (designated as MEM2×) was 2×MEM supplemented with FBS (10%, v/v), 4 mM L-glutamine, and 200 U/mL penicillin and 200 μg/mL streptomycin. It is favorable to use a medium lacking phenol red due to eventual color interference with later neutral red staining. The traditional agar overlay medium (designated as 1×MEM_{AO}) was MEM_{2×} supplemented with 0.33% neutral red and 4% agar (Sigma-Aldrich, St. Louis, Mo.) to make final concentration of 0.01% neutral red and 1% agar. Base agar of the two layer modified/embedded modified agar overlay (designated as 1×MEM_{AOM}) was MEM_{2×} supplemented with 2.5% Bacto agar (BD, Franklin Lakes, N.J.) and 1×MEM_{05FBS} to make final concentration of 0.5% Bacto agar.

L929 Cell Culture and Maintenance.

Mouse fibroblast L929 cells were obtained from the American Type Culture Collection (ATCC, Manassas, Va.) and expansively cultured to make master stocks (within 20 passages) maintained in the liquid nitrogen. Cells were confirmed free from mycoplasma by ATCC. After thawing, L929 cells were grown at 37° C. with a 5% (v/v) carbon dioxide (CO₂) humidified atmosphere to achieve logarithmic growth. The cells were routinely diluted to ˜1×10⁵ cells/mL in growth media (1×MEM_{10FBS}) to prevent overgrowth (>2×10⁶ cells/mL) and were used after two weeks of passage and during logarithmic growth.

### Data Analysis

Determination of cytotoxicity and test acceptance criteria for modified agar overlay method followed those described in USP and ISO 10993-5 (ISO10993-5, 2009; USP, 2017): 1) negative controls should not have detectable zone beyond the area under specimen (grade ≤2), and 2) positive controls must have produced a zone extending specimen size ≥1.0 cm (reactivity moderate to severe) (Table 1). The test article meets the ‘pass’ criteria of the test if the biological response is less than or equal to grade 2 (mild).

TABLE 1
Reactivity grades for modified agar overlay method.

| Grade | Reactivity | Cytotoxicity | Description of reactivity zone |
|---|---|---|---|
| 0 | None | non-cytotoxic | No detectable zone around or under specimen |
| 1 | Slight | non-cytotoxic | Some malformed or degenerated cells under specimen |
| 2 | Mild | non-cytotoxic | Color zone with cells limited to area under specimen |
| 3 | Moderate | cytotoxic | Color zone with cells extending specimen size up to 1.0 cm |
| 4 | Severe | cytotoxic | Color zone with cells extending farther than 1.0 cm beyond |

### Example 1

Direct Contact.

L929 Cells were seeded (2 mL/Well) at 1×10⁵ Cells/mL, Incubated for 48 Hours (at 37° C.) and then medium was replaced with 0.8 mL of 1×MEM_{05FBS}. Wako disc and reference material C were the negative controls; and latex, reference material A, and B were the positive controls. The paper filter discs (20 μL of either 100% ethanol or 2000 ppm (0.2%) Benzalkonium Chloride (BAK)) were also evaluated. At the end of the 24-hour incubation, all materials and medium were removed and cells were stained with 0.1% Trypan blue (Sigma-Aldrich, St. Louis, Mo.), examined by phase contrast microscopy, and the potential cytotoxicity of each test material is graded according to ISO/USP guidelines (USP, 2017).

Positive (reference materials A, B, and latex) and negative controls (Wako disc and reference material C) were used in this study. All the positive controls are cytotoxic and negative controls are non-cytotoxic (Table 2). Both Wako disc and reference material C had slight reactivity indicated that the physical movement of test articles cannot be fully prevented during the study even though the volume of culture medium was reduced to 0.8 mL/well.

TABLE 2
Comparison of the reactivity grades from direct contact,
agar overlay, and modified agar overly methods^{a}.

| | | Direct | Two | Two layer |
|---|---|---|---|---|
| | | contact | layer | embedded |
| | ISO/USP | (without | modified | modified |
| | agar | agar | agar | agar |
| Test articles | overlay | layer) | overlay | overlay |
| Wako | 0 | ≤1 | 0 | 0 |
| (Negative, High | (non- | (non- | (non- | (non- |
| Density) | cytotoxic) | cytotoxic) | cytotoxic) | cytotoxic) |
| RM-C | 0 | ≤1 | 0 | 0 |
| (Negative, High | (non- | (non- | (non- | (non- |
| Density) | cytotoxic) | cytotoxic) | cytotoxic) | cytotoxic) |
| RM-A | 3 | 4 | 4 | 4 |
| (Strong Positive) | (cytotoxic) | (cytotoxic) | (cytotoxic) | (cytotoxic) |
| RM-B | 3 | 4 | 4 | 4 |
| (Weak Positive) | (cytotoxic) | (cytotoxic) | (cytotoxic) | (cytotoxic) |
| Latex | ≤3 | 4 | 4 | ≥3 |
| (Strong Positive) | (cytotoxic/ | (cytotoxic) | (cytotoxic) | (cytotoxic) |
| | non- | | | |
| | cytotoxic) | | | |
| Ethanol | 0 | ≤3^{b} | 0 | 2 |
| (100%) | (non- | (cytotoxic/ | (non- | (non- |
| | cytotoxic) | non- | cytotoxic) | cytotoxic) |
| | | cytotoxic) | | |
| BAK | 0 | 4^{b} | 3 | 3 |
| (2000 ppm) | (non- | (cytotoxic) | (cytotoxic) | (cytotoxic) |
| | cytotoxic) | | | |

^{a}Data represent three independent studies and each study has three replicates.
^{b}Data represent 20 μL of test articles in 800 μL of medium

### Example 2: ISO/USP Agar Overlay

L929 cells were prepared as described in the direct contact method except that medium was replace with 2 mL of agar overlay medium (1×MEM_{AO}) after 48 hours incubation (at 37° C.). All the positive, negative, and paper filter discs (100 μL of either 100% Ethanol or 2000 ppm (0.2%) BAK) were placed on top of the solidified agarose surface in each well. After 24 hours of incubation, cells were examined macroscopically to observe cell decolorization around the test materials for the determination cell lysis zone. After macroscopic examination, cells were examined microscopically to verify any decolorized zones and to determine cell morphology in proximity to the article.

Results with both reference materials A and B had lower reactivity as compared to the direct contact method (Tables 2 and 3). In contrast to direct contact method, Wako disc and reference material C had no reactivity in the agar overlay method because of the protection of the agar layer. The lack of cytotoxicity observed with latex during the study might due to the uneven thickness and powder distribution on top of the gloves. This phenomenon might make the latex as an unfavorable positive control for agar overlay and should be used with cautious. For the liquid test articles, BAK and Ethanol were evaluated (Table 2) and both materials were not cytotoxic (grade 0) in the traditional agar overlay method. These results are consistent with previous observations that BAK failed to show cytotoxicity responses using the traditional agar overlay method at a concentration known to be toxic to the rabbit eyes (Bruinink and Luginbuehl, 2012). BAK (100 μL of 0.2% BAK was added onto either 2.6 mL or 4.0 mL of cell/agar mix) was chosen for this study because it is a common preservative used in various medical preparations ranging in concentrations from 0.01% to 0.05%.

TABLE 3
Examples of color zone formation for solid materials.

| | | | Two layer |
|---|---|---|---|
| | | Two layer | embedded |
| | ISO/USP | modified | Modified |
| | Agar | Agar | Agar |
| Test Articles | Overlay | Overlay | Overlay |
| Cell only- | Grade 0 | Grade 0 | Grade 0 |
| no treatment | | | |
| Negative-Wako Disc | Grade 0 | Grade 0 | Grade 0 |
| Negative-Reference | Grade 0 | Grade 0 | Grade 0 |
| Material C | | | |
| Strong Positive- | Grade 3; color | Grade 4; | Grade 4; |
| Reference | zone | color zone | color zone |
| Material A | extended ≤ | extended > | extended > |
| | 1.0 cm | 1.0 cm) | 1.0 cm |
| Weak Positive- | Grade 3; color | Grade 4; | Grade 4; |
| Reference | zone | color zone | color zone |
| Material B | extended ≤ | extended > | extended > |
| | 1.0 cm | 1.0 cm | 1.0 cm |
| Latex | Grade 3; | Grade 4; | Grade 3; |
| | color zone | color zone | color zone |
| | extended ≤ | extended > | extended ≤ |
| | 1.0 cm) | 1.0 cm | 1.0 cm |

### Example 3

Modified and Embedded Modified Agar Overlay.

1×MEM_{AOM} was used as the nutrient supplement agar at 2 mL/well.

A) Two layer modified agar overlay. After the nutrient agar solidified, L929 cells (5.4×10⁶ cells/mL) were mixed with 0.33% neutral red at a 1:32 ratio (v/v) and 1×MEM_{AOM} at 1:2 ratio (v/v) to make 0.01% neutral red and 1.8×10⁶ cells/mL cell agar mixture. Then, 600 μL of this cell agar mixture was added on top of the base agar with a final cell concentration of 1.0×10⁶ cells/well. Test materials were loaded as described in the ISO/USP agar overlay method.

B) Two layer embedded modified agar overlay. The test materials were added after the base nutrient agar solidified, and 2.0 mL of L929 (1.8×10⁶ cells/mL) cell agar mixture described above was added on top of the test materials to fully cover the test materials with a final cell concentration of 3.6×10⁶ cells/well. After 24 hours of incubation, cells were examined as described in ISO/USP agar overlay method.

C) For the extended study duration, cells were prepared as described in A) and B). Reference material A and no treatment were evaluated for up to 72-hour incubation.

D) two layer modified and embedded modified agar overlay for liquid materials. Paper filter discs (100 μL of either 100% Ethanol or 2000 ppm BAK) were prepared as described in A) and B).

In comparing with the direct contact method (Table 2 and 3), the results of modified agar overlay method showed equal sensitivity for all the positive controls. In addition, the two negative controls used in this study showed non-reactivity and thus the potential physical damage by the overlaying test materials was avoided. For the liquid test articles (Table 4), ethanol was chosen to evaluate the potential feasibility for testing the volatile materials and was non-cytotoxic (grade 0) in the modified agar overlay method but mild cytotoxic (grade 2) in the embedded modified agar overlay method. This non-cytotoxicity response might due the ethanol concentration in the embedded approach (100 μL on paper filter with 2 mL of cells on the top) was too low. BAK was grade 3 (moderate cytotoxic) in both embedded and modified agar overlay method.

TABLE 4
Examples of color zone formation for liquids.

| | | Two layer | Two layer |
|---|---|---|---|
| | ISO/USP | modified | embedded |
| | Agar | Agar | Modified |
| Test Articles | Overlay | Overlay | Agar Overlay |
| Negative-PBS | Grade 0 | Grade 0 | Grade 0 |
| 100 μL of PBS added on | | | |
| top of paper filter | | | |
| Positive-Ethanol | Grade 0 | Grade 0 | Grade 2; |
| 100 μL of 100% ethanol | | | no zone |
| added on top of paper | | | but color |
| filter | | | changed |
| | | | mild |
| Positive-BAK | Grade 0 | Grade 3; | Grade 3; |
| 100 μL of 2000 | | no zone but | no zone |
| ppm BAK added | | color | but color |
| on top of paper | | changed | changed |
| filter | | moderate | moderate |

Since cells in semisolid medium are lack of contact inhibition, we further investigated the potential extended study duration using reference material A. Reference material A showed increased cytotoxicity at 72 hours as compared to the 24 hours treatment and no signs of cytotoxicity for cell only treatment (Table 5). All these results suggest that this modified agar overlay approach could increase overall sensitivity, might have the potential to detect volatile materials, and be used for extended study duration.

TABLE 5
Evaluation of the potential for modified agar
overlay extended study duration

| | Two layer | Two layer |
|---|---|---|
| | modified | embedded |
| | Agar | Modified Agar |
| Treatments | Overlay | Overlay |
| 24 hours | Grade 0 | Grade 0 |
| Cell only | | |
| 24 hours | Grade 4 | Grade 4 |
| Reference material A | | |
| 48 hours | Grade 0 | Grade 0 |
| Cell only | | |
| 48 hours | Grade 4 | Grade 4 |
| Reference material A | | |
| 72 hours | Grade 0 | Grade 0 |
| Cell only | | |
| 72 hours | Grade 4 | Grade 4 |
| Reference material A | | |

### DISCUSSION AND CONCLUSIONS

Due to the increased new types of medical devices to be evaluated, there is a need to develop new methodologies which might better represent the condition for patient use. The goal was to develop a sensitive qualitative direct contact method for new medical device materials such as adhesives and devices prepared in situ. Our preliminary results demonstrated that this modified agar overlay assay described in this invention increased overall sensitivity due to the decreased agar volume and agar concentration as compared to the ISO/USP agar overlay method. In addition, this method avoids the physical trauma caused by unintentional movement/pressure of the test material to the adhesion cells commonly observed in the direct contact method. The major critical factors for this modified agar overlay method development include: 1) using a tightly controlled, well characterized, and consistent source of cells, and 2) performing appropriate agar layer thickness, agar concentration and incubation time for each experimental model system. Growth of cells in semisolid medium has been used as a gold-standard assay for carcinogenesis system to measure cellular transformation in vitro.

While there are a number of cell lines that can be used, L929 and Chinese hamster lung (V79) cell lines are preferred for standardized cytotoxicity in this patent application. Both cell lines are commonly recommended and routinely used in in vitro cytotoxicity assessment and have the ability to grow in semisolid medium. The data demonstrated that L929 cells have similar sensitivity and can be used in this modified agar overly method. One disadvantage of using traditional agar overlay with monolayer adhesion cells is that normal cells will stop replicating when the cells occupy the entire substratum. At this point, the cell suspension in semi-solid agar approach might be able to avoid contact inhibition and thus provide extended test duration. Finally, the neutral red stain itself might be able to cause cytotoxicity or interact with some chemicals to form zones which could be mistaken for a cytotoxicity response, and thus cells/neutral red incubation time is not recommend for over 24 hours. In this new modified agar overlay approach, significantly lower neutral red concentration (neutral red is included only in the top cell agar mix) was used and the results (cell only and reference material A) showed that cells/neutral red incubation time could be extended to 72 hours.

In summary, the modified agar overlay test is highly reproducible, very easy to conduct, and overcomes many limitations of the direct contact and traditional agar overlay methods (Table 6). The embedded modified approach renders it possible to not only investigate the effect on regular test material but also to assess the potential volatiles which might not be caught by traditional agar overlay or extract methods using paper filter discs. This method represents a valuable addition to the battery of assays for detection of potentially toxic materials leaching from a test material that is either a solid or liquid.

TABLE 6
Summary of the advantages and disadvantages of the modified agar overlay method.

| Assays | Advantages | Disadvantages |
|---|---|---|
| Two layer | Cells are protected from | Like all the qualitative assays, modified |
| modified and two | physical movement. | agar overlay method might be |
| layer embedded | Cells are closer to the materials | subjective and could be analyst |
| Modified Agar | as compared to the agar | dependent. |
| Overlay | overlay method. | The potential cytotoxic leachates may |
| | Leachates are able to diffuse in | bind to the agar (0.3%) and not be able |
| | the diluted soft agar (0.3%). | to diffuse across the plate as |
| | Might be used for adhesives. | compared to the liquid medium; thus, |
| | Might be able to detect volatile | the cells are not fully exposed to the |
| | materials | test substance. |
| | Might be able to have extended | Might be more suitable for solid |
| | study duration due to the lack of | materials as liquid materials could |
| | the contact inhibition | further dilute the agar and damage the |
| | phenomena which is observed | agar integrity. |
| | in the direct and traditional agar | |
| | overlay methods using | |
| | adhesion cells. | |
| | A quicker turnaround time for | |
| | results due to the immediate | |
| | exposure of test article after the | |
| | cell agar mixture solidified. | |
| | Neutral red dye might not be | |
| | required. | |
| Traditional Agar | Cells are protected from | The potential cytotoxic leachates may |
| Overlay | physical/mechanical damage | bind to the agar (1%) and not be able |
| | due to movement of the test | to diffuse across the plate as |
| | article. | compared to the liquid medium; thus, |
| | Cells need to reach sub | the cells are not fully exposed to the |
| | confluency (80%) condition | test substance. |
| | before any treatment (normally | Might not be suitable for volatile |
| | takes 48 hours before test | materials. |
| | article treatment). | Neutral red could cause cytotoxicity |
| | | and cell/neutral red incubation might |
| | | not be over 24 hours. |
| | | Not suitable for extended study |
| | | duration due to the contact inhibition |
| | | phenomena. |
| Direct Contact | It is recommended for leachable | The cells in the direct contact are more |
| | substances that may be | susceptible to potential mechanical |
| | deactivated during extraction or | damage by the overlying. |
| | for devices or materials that | The test material is either placed |
| | come in direct contact with | directly on top of the cells which might |
| | tissue (e.g., eye contact). | cause damages due to lacking of |
| | Cells need to reach sub | nutrient and O₂/CO₂ or the cells are |
| | confluency (80%) condition | cultured on the surface of the medical |
| | before any treatment (normally | device itself, although usefulness of |
| | takes 48 hours before test | the latter is limited by the ability of the |
| | article treatment). | cells to adhere to the test material. |
| | | Not suitable for extended study |
| | | duration due to the contact inhibition |
| | | phenomena. |

The invention has been described with the aid of a specific embodiment of the process or apparatus, respectively. However, the invention is not limited to the specific embodiment described but rather various changes and modifications are possible without departing from the general concept underlying the invention. Therefore, the scope of protection is defined by the appended claims.

What is claimed is:

1. A method of measuring the cytotoxicity of a medical device, the method comprising:
a) providing a first agar-nutrition medium to form a first layer onto a base surface of a well plate;
b) placing a mixture of second agar-nutrition medium and a cell to form a second layer onto the top of the first layer, wherein the second agar-nutrition medium comprising a color dye;
c) placing the medical device onto the top of the second layer;
d) incubating the well plate containing the medical device with the first agar-nutrition medium and the mixture of second agar-nutrition medium and a cell;
e) determining size of zone of decolorization around and under the medical device;
wherein the first agar-nutrition medium has a higher agar concentration than the second agar-nutrition medium;
wherein each of the first agar-nutrition medium and the second agar-nutrition medium has the agar concentration less than 2 percent.

2. The method of claim 1, wherein the cell is a L929 cell or a V79 cell.

3. The method of claim 1, wherein the nutrition medium in the first agar-nutrition medium comprising a minimal essential medium (MEM1) supplemented with 2 mM L-glutamine, 100 U/mL penicillin and 100 mg/mL streptomycin, and a fetal bovine serum, wherein the fetal bovine serum concentration is 5% (v/v) or 10% (v/v).

4. The method of claim 1, wherein the first agar-nutrition medium comprising a color dye.

5. The method of claim 4, wherein the color dye in the first agar-nutrition medium and the second-agar nutrition medium is a red dye.

6. The method of claim 1, wherein the step of incubating is for 24 hours at a temperature of 37° C.

7. The method of claim 1, wherein each of the first agar-nutrition medium and the second agar-nutrition medium has the agar concentration less than 1.5 percentage

8. The method of claim 7, wherein each of the first agar-nutrition medium and the second agar-nutrition medium has the agar concentration less than 1.0 percentage

9. The method of claim 8, wherein each of the first agar-nutrition medium and the second agar-nutrition medium has the agar concentration less than 0.75 percentage.

10. The method of claim 9, wherein the first agar-nutrition medium has the agar concentration from 0.5 to 0.7 percent and the second agar-nutrition medium has the agar concentration from 0.3 to 0.49 percent.

11. A method of testing cytotoxicity of a medical device comprising:
a) providing a first agar-nutrition medium to form a first layer onto a base surface of a well plate;
b) placing the medical device onto the top of the first layer;
c) placing a mixture of second agar-nutrition medium and a cell onto the top of the medical device and the first layer to form a second layer to fully cover the medical device, wherein the second agar-nutrition Medium comprising a color dye;
d) incubating the well plate containing the medical device with the first agar-nutrition medium and the mixture of second agar-nutrition medium and a cell; and
e) determining size of zone of decolorization around the medical device;
wherein the first agar-nutrition medium has a higher agar concentration than the second agar-nutrition medium;
wherein both the first agar-nutrition medium and the second agar-nutrition medium have the agar concentration less than 2 percent.

12. The method of claim 11, wherein the cell is a L929 cell or a V79 cell.

13. The method of claim 11, wherein the nutrition medium in the first agar-nutrition medium comprising a minimal essential medium (MEM1) supplemented with 2 mM L-glutamine, 100 U/mL penicillin and 100 mg/mL streptomycin, and a fetal bovine serum, wherein the fetal bovine serum concentration is 5% (v/v) or 10% (v/v).

14. The method of claim 11, wherein the first agar-nutrition medium comprising a color dye.

15. The method of claim 14, wherein the color dye in the first agar-nutrition medium and the second agar-nutrition medium is a red dye.

16. The method of claim 11, wherein the step of incubating is for 24 hours at a temperature of 37° C.

17. The method of claim 11, wherein each of the first agar-nutrition medium and the second agar-nutrition medium has the agar concentration less than 1.5 percent

18. The method of claim 17, wherein each of the first agar-nutrition medium and the second agar-nutrition medium has the agar concentration less than 1.0 percent

19. The method of claim 18, wherein each of the first agar-nutrition medium and the second agar-nutrition medium has the agar concentration less than 0.75 percent

20. The method of claim 19, wherein the first agar-nutrition medium has the agar concentration from 0.5 to 0.7 percent and the second agar-nutrition medium has the agar concentration from 0.3 percent to 0.40 percent.
